(19) United States
(12) Patent Application Publication
(10) Pub. No.: US 2018/0282799 A1
(43) Pub. Date: Oct. 4, 2018

(54) TARGETED LOCUS AMPLIFICATION USING CLONING STRATEGIES

(71) Applicant: Koninklijke Nederlandse Akademie van Wetenschappen, Amsterdam (NL)

(72) Inventors: Wouter Leonard DE LAAT, Doorn (NL); Carlo VERMEULEN, Nieuwegein (NL)

(73) Assignee: Koninklijke Nederlandse Akademie van Wetenschappen, Amsterdam (NL)

(21) Appl. No.: 15/765,741

(22) PCT Filed: Oct. 5, 2016
(86) PCT No.: PCT/NL2016/050686

§ 371 Date: Apr. 4, 2018

(30) Foreign Application Priority Data
Oct. 5, 2015 (NL) 2015569

(51) Int. Cl. C12Q 1/6853; C12N 15/66; C12Q 1/6869; C12Q 1/686; C12N 15/10

(52) U.S. Cl. CPC C12Q 1/6853; C12N 15/66; C12Q 1/6869; C12Q 2523/101; C12N 15/10; C12Q 2521/501; C12Q 1/686

(57) ABSTRACT

The current invention relates to strategies for selection and amplification of genomic regions of interest. The strategy involves an amplification step in a host cell. A target nucleotide sequence associated with the genomic region of interest is used to selectively provide a DNA circles derived from the genomic region of interest with an origin of replication and a selection gene. This is in particular useful i.a. for determining DNA sequences of a genomic region of interest, for use in building contiguous sequences and/or for DNA mapping.

### FIELD OF THE INVENTION

The present invention relates to the field of molecular biology and more in particular to DNA technology. The invention relates to strategies for determining DNA sequences of a genomic region of interest, for use in building contiguous sequences and/or for DNA mapping. In particular the present invention relates to selection and amplification of genomic regions of interest.

### BACKGROUND

Considerable effort has been devoted to develop “target enrichment” strategies for sequencing, in which genomic regions from a DNA sample are selectively captured and/or selectively amplified and subsequently sequenced (reviewed in Mamanova et al., Nature Methods, 2010, (2):111-118). One of the recent developments for target enrichment involves targeted proximity ligation amplification methods (TPLA) like target locus amplification (TLA) and 4C. In these technologies DNA is fragmented and DNA fragments that originally were close together on the linear DNA template (e.g. the chromosome) remain in each other's physical proximity, e.g. because the DNA that was fragmented was in a crosslinked state. Subsequently, the DNA fragments are ligated. Because the DNA fragments are in each other's proximity, e.g. in a crosslinked state, the subsequent DNA ligation step favours the ligation of DNA fragments that are in each other's proximity, also termed proximity ligation. For targeted locus amplification typically a target nucleotide sequence in or near a genomic region of interest is selected. The DNA fragment comprising the target nucleotide sequence is ligated to DNA fragments that are in its proximity, i.e. that represent the genomic region of interest. By amplifying ligated DNA fragments that only comprise the target nucleotide sequence, ligated DNA fragments that represent the genomic region of interest are amplified. Such amplification comprises primer based amplification methods such as a polymerase chain reaction wherein primers are designed to hybridize with the target nucleotide sequence. Targeted proximity ligation amplification methods are described i.a. in WO2012005595, WO 2007/004057 and WO 2008/08845.

US2002/0150945 describes the random integration of origins of replication in the intact genome of living cells and subsequent selection of cells with a given phenotype, for the purpose of analysing the genomic context responsible for the given phenotype and subsequent generation of knock outs. The approach requires the use of transgenic cells that have been provided with origins of replication at random genomic locations, i.e. without the use of and without any prior understanding of a target nucleotide sequence.

### SUMMARY OF THE INVENTION

The inventors, using methods to selectively isolate (by selective capture or selective amplification) and sequence genomic regions from a DNA sample, faced the problem that most current methods require prior knowledge of two target nucleotide sequences to enable isolation of the intervening genomic region, being the genomic region that is flanked by these two target nucleotide sequences. Examples of such methods include the isolation of Yeast Artificial Chromosomes and Bacterial Artificial Chromosomes (YACs and BACs), the TAR method (Transformation Associated Recombination) (Kouprina N, Larionov V. TAR cloning: insights into gene function, long-range haplotypes and genome structure and evolution. Nat Rev Genet. 2006 October; 7(10):805-12) and variants thereof such as CRIPSR-Cas mediated TAR (Lee NC1, Larionov V1, Kouprina N. Highly efficient CRISPR/Cas9-mediated TAR cloning of genes and chromosomal loci from complex genomes in yeast. Nucleic Acids Res. 2015 Apr. 30; 43(8):e55. doi: 10.1093/nar/gkv112) and Jiang W, Zhao X, Gabrieli T, Lou C, Ebenstein Y, Zhu T F. Cas9-Assisted Targeting of Chromosome segments CATCH enables one-step targeted cloning of large gene clusters. Nat Commun. 2015 Sep. 1; 6:8101. doi: 10.1038/ncomms9101. Disadvantage of such strategies that are dependent on two dispersed target nucleotide sequences to isolate the intervening genomic region of interest is that the methodologies will fail isolating the genomic region of interest in cases when one or both of the target nucleotide sequences is no longer present or no longer intact, and in cases when chromosomal rearrangements have placed them too far apart. The target locus amplification method does not face this problem, but faces specific problems associated with the use of primer based amplification methods, such as PCR. For example, PCR inherently introduces sequence-dependent amplification biases. Examples of difficult to PCR-amplify sequences (well known in the field) are low complexity sequences, repetitive sequences, AT-rich sequences GC-rich sequences. Furthermore, PCR amplification can introduce a size-dependent bias. For example, when DNA circles (see FIG. 1) are to be amplified, the simultaneous amplification of both smaller and larger DNA circles comprising the DNA fragment with the target nucleotide sequence will result in a much more efficient amplification of the smaller DNA circles as compared to the larger DNA circles. Also, PCR amplification of larger sequences composed of larger ligated DNA fragments or of concatemers of multiple ligated DNA fragments is highly inefficient. This can occur with sequences larger than 2 kilobases, and certainly occurs for those larger than 3 kilobases, particularly if the to be amplified DNA mixture also contains smaller concatemers. As said, PCR amplification of large concatemers composed of many different ligated DNA fragments is not possible. Amplification of large concatemers is important to obtain maximal sequence information from each fragmented crosslinked DNA aggregate. Amplification of large concatemers also facilitates haplotyping. For example if the amplified material is analysed on a sequencing platform that enables analysis of long sequence reads (e.g. as available from PacBio or Oxford Nanopore). Furthermore, the inventors also realized that similar issues also play a role for selection and amplification of genomic regions of interest that do not involve targeted proximity ligation amplification.

Hence, the current inventors devised strategies for selection and amplification of genomic regions of interest that comprise a (single) target nucleotide sequence and an amplification step in a host cell, wherein the target nucleotide sequence associated with the genomic region of interest is used to selectively provide a DNA circle with an origin of replication and a selection gene. This way, the current invention enables selective isolation and sequencing of a genomic region of interest that resides on either side of a single target nucleotide sequence. In this invention, it does not matter what the nucleotide sequence composition of the genomic region of interest is. Even if its sequence composition is not as predicted from the reference genome, which may occur in patients because of mutations, amplifications, deletions, inversions or other types of chromosomal rearrangements, the current invention will enable isolation and sequencing of the genomic region of interest as long as the, preferably single, target nucleotide sequence is present. The current invention avoids the dependency on two target nucleotide sequences to isolate and analyse a genomic region flanked by these two target nucleotide sequences, having all the restraints as listed above. Also, instead of an enzymatic replication which has all the restraints as listed above, the host cell can amplify the DNA circles comprising the target nucleotide sequence, while DNA circles not carrying the target nucleotide sequence are not amplified. Amplification in host cells has the benefit that they can accommodate DNA circles that are large. For example, as said PCR amplification of DNA sequences larger than 2-3 kilobases can be inefficient and difficult, molecular biology cloning of DNA plasmids in bacteria can involve the use of DNA sequences ranging in size from 3 kilobases to 250 kilobases. Furthermore, the DNA replication machinery of the host cell is more reliable as it is less error prone and it readily amplifies low complexity sequences, repetitive sequences, AT-rich sequences GC-rich sequences as these naturally occur in the genome of the host cell. Hence, by carrying out amplification in host cells, one or more of the constraints as listed above for primer based amplifications can be reduced or even overcome. For example, amplification in bacteria is much less susceptible to the introduction of sequence-dependent amplification biases. Amplification restrictions due to size-biases is much less in host cells, e.g. bacteria, as compared to PCR. Host cells can also easily accommodate large DNA molecules, this allows the number of analysable ligated fragments to increase per given amount of starting material (e.g. amount of cells) to thereby increase the coverage that can be obtained. Furthermore, as bacterial clones can be selected and analysed that contain individual large DNA molecules, haplotyping is facilitated.

Hence, the method of the invention relates to selection and amplification of a genomic region of interest, wherein the genomic region of interest contains a (preferably) single target nucleotide sequence, wherein the method comprises:
providing DNA circles derived from genomic DNA, wherein said DNA circles comprise DNA circles containing the single target nucleotide sequence;
editing the target nucleotide sequence such that the DNA circles containing the target nucleotide sequence are provided selectively with an origin of replication sequence and a selection gene;
transferring the DNA circles comprising the DNA circles containing said origin of replication sequence and said selection gene into a host cell; and
selectively culturing host cells comprising DNA circles containing said origin of replication sequence and said selection gene.

The DNA circles derived from genomic DNA may be DNA circles such as can be generated in targeted proximity ligation amplification methods, i.e. DNA circles are formed wherein each circle comprises several DNA fragments that are derived from the genome. The DNA circles derived from genomic DNA may also be derived from single genomic DNA fragments, i.e. wherein one DNA circle is then derived from a single contiguous DNA fragment as for example the large 30-200 kb DNA fragments typically obtained by genomic DNA isolation procedures (in contrast to the DNA circles that are a composite of many DNA fragments as in targeted proximity ligation amplification methods). The generation of DNA circles combined with a target nucleotide sequence has the benefit that only little DNA sequence information is required, i.e. only sequence information of a target nucleotide sequence that represents or locates inside the genomic region of interest is required. In the DNA circles, sequences flanking the target nucleotide sequence that represent the genomic region of interest (as e.g. comprised in a single genomic DNA fragment that was comprised in a DNA circle, or e.g. as comprised of the several ligated DNA fragments that were comprised in a DNA circle), are amplified. The genomic region of interest may be up to 300 kb in size, whereas the target nucleotide sequence may be as small as about 30 nucleotides. The target nucleotide sequence that is selected is preferably unique within the context of the genome, i.e. it is preferably selected such that only DNA circles with the genomic region of interest are provided with said origin of replication and selection gene. By using the methods of the invention it is now possible to selectively amplify a genomic region of interest wherein the amplified products may not suffer from one or more of the drawbacks such as listed above.

Definitions In the following description and examples, a number of terms are used. In order to provide a clear and consistent understanding of the specification and claims, including the scope to be given such terms, the following definitions are provided. Unless otherwise defined herein, all technical and scientific terms used have the same meaning as commonly understood by one of ordinary skill in the art to which this invention belongs. The disclosures of all publications, patent applications, patents and other references are incorporated herein in their entirety by reference.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N.Y., 1989; Ausubel et al., Current Protocols in Molecular Biology, John Wiley & Sons, New York, 1987 and periodic updates; and the series Methods in Enzymology, Academic Press, San Diego.

As used herein, the singular forms “a,” “an” and “the” include plural referents unless the context clearly dictates otherwise. For example, a method for isolating “a” DNA molecule, as used above, includes isolating a plurality of molecules (e.g. 10's, 100's, 1000's, 10's of thousands, 100's of thousands, millions, or more molecules).

A “genomic region of interest” according to the invention is a DNA sequence of an organism of which it is desirable to determine, at least part of, the DNA sequence. For instance, a genomic region which is suspected of comprising an allele associated with a disease may be a genomic region of interest.

As used herein, the term “allele(s)” means any of one or more alternative forms of a gene at a particular locus. In a diploid cell of an organism, alleles of a given gene are located at a specific location, or locus (loci plural) on a chromosome. One allele is present on each chromosome of the pair of homologous chromosomes. Thus, in a diploid cell, two alleles and thus two separate (different) genomic regions of interest may exist.

A “haplotype” according to the invention is a set of DNA sequence variations, including but not limited to single-nucleotide polymorphisms (SNPs), structural variations such as (micro)deletions or—insertions or repeat length variations that are attributed to a single chromosome (e.g. a single allele corresponding to a genomic region of interest). Hence, for a diploid genome DNA sequence variations can be determined and for each of the two homologous chromosomes a contig may be built, and/or a haplotype can be made.

A “nucleic acid” according to the present invention may include any polymer or oligomer of pyrimidine and purine bases, preferably cytosine, thymine, and uracil, and adenine and guanine, respectively (See Albert L. Lehninger, Principles of Biochemistry, at 793-800 (Worth Pub. 1982) which is herein incorporated by reference in its entirety for all purposes). The present invention contemplates any deoxyribonucleotide, ribonucleotide or peptide nucleic acid component, and any chemical variants thereof, such as methylated, hydroxymethylated or glycosylated forms of these bases, and the like. The polymers or oligomers may be heterogeneous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

A “sample DNA” is a sample that is obtained from an organism or from a tissue of an organism, or from tissue and/or cell culture, which comprises genomic DNA. Genomic DNA encodes the genome of an organism that is the biological information of heredity which is passed from one generation of an organism to the next. A sample DNA from an organism may be obtained from any type of organism, e.g. micro-organisms, viruses, plants, fungi, animals, humans and bacteria, or combinations thereof. For example, a tissue sample from a human patient suspected of a bacterial and/or viral infection may comprise human cells, but also viruses and/or bacteria. The sample may comprise cells and/or cell nuclei. The sample DNA may be from a patient or a subject who may be at risk or suspected of having a particular disease, for example cancer or any other condition which warrants the investigation of the DNA of the organism.

With “crosslinking” according to the invention is meant reacting DNA at two different positions, such that these two different positions may be connected. The connection between the two different positions may be directly, forming a covalent bond between DNA strands. Two DNA strands may be crosslinked directly using UV-irradiation, forming covalent bonds directly between DNA strands. The connection between the two different positions may be indirectly, via an agent, e.g. a crosslinker molecule. A first DNA section may be covalently connected to a first reactive group of a crosslinker molecule comprising two reactive groups, that second reactive group of the crosslinker molecule may be covalently connected to a second DNA section, thereby crosslinking the first and second DNA section indirectly via the crosslinker molecule. A crosslink may also be formed indirectly between two DNA strands via more than one molecule. For example, a typical crosslinker molecule that may be used is formaldehyde. Formaldehyde induces covalent protein-protein and DNA-protein crosslinks. Formaldehyde thus may crosslink different DNA strands to each other via their associated proteins. For example, formaldehyde can react with a protein and DNA, covalently connecting a protein and DNA via the crosslinker molecule. Hence, two DNA sections may be crosslinked using formaldehyde forming a connection between a first DNA section and a protein, the protein may form a second connection with another formaldehyde molecule that connects to a second DNA section, thus forming a crosslink which may be depicted as DNA1-crosslinker-protein-crosslinker-DNA2. In any case, it is understood that crosslinking according to the invention may comprise forming covalent connections (directly or indirectly) between strands of DNA that are in physical proximity of each other. DNA strands may be in physical proximity of each other in the cell, as DNA is highly organised, while being separated from a sequence point of view e.g. by 100 kb. As long as the crosslinking method is compatible with subsequent fragmenting and ligation steps, such crosslinking may be contemplated for the purpose of the invention.

A “sample of crosslinked DNA” is a sample DNA which has been subjected to crosslinking. Crosslinking the sample DNA has the effect that the three-dimensional state of the genomic DNA within the sample remains largely intact. This way, DNA strands that are in physical proximity of each other remain in each other's vicinity. A “sample of crosslinked DNA” may also be reconstituted chromatin that has been crosslinked, wherein genomic DNA that has been isolated from a cell (e.g. a tissue sample or a DNA sample) is subjected to chromatin reconstitution or otherwise packaged or coated by proteins or molecules that facilitate crosslinking, and subsequent crosslinking. A sample of crosslinked DNA comprises genomic DNA.

“Reversing crosslinking” according to the invention comprises breaking the crosslinks such that the DNA that has been crosslinked is no longer crosslinked and is suitable for subsequent steps such as ligation, amplification and/or sequencing steps. For example, performing a protease K treatment on a sample DNA that has been crosslinked with formaldehyde will digest the protein present in the sample. Because the crosslinked DNA is connected indirectly via protein, the protease treatment in itself may reverse the crosslinking between the DNA. The protein fragments that remain connected to the DNA may hamper subsequent sequencing and/or amplification. Hence, reversing the connections between the DNA and the amino acids in the protein may also result in “reversing crosslinking”. The DNA-crosslinker-protein connection may be reversed through a heating step for example by incubating at 70° C. As in a crosslinked DNA large amounts of protein can be present, it is often desirable to digest the protein with a protease in addition. Hence, any “reversing crosslinking” method may be contemplated wherein the DNA strands that are connected in a crosslinked sample no longer are connected and become suitable for sequencing and/or amplification.

“Fragmenting DNA” includes any technique that, when applied to DNA, which may be crosslinked DNA or not, or any other DNA, results in DNA fragments. Techniques well known in the art are sonication, shearing and/or enzymatic restriction, but other techniques can also be envisaged.

A “restriction endonuclease” or “restriction enzyme” is an enzyme that recognizes a specific nucleotide sequence (recognition site) in a double-stranded DNA molecule, and will cleave both strands of the DNA molecule at or near every recognition site, leaving a blunt or a 3′- or 5′-overhanging end. The specific nucleotide sequence which is recognized may determine the frequency of cleaving, e.g. a nucleotide sequence of 6 nucleotides occurs on average every 4096 nucleotides, whereas a nucleotide sequence of 4 nucleotides occurs much more frequently, on average every 256 nucleotides.

“Ligating” according to the invention involves the joining of separate DNA fragments. The DNA fragments may be blunt ended, or may have compatible overhangs (sticky overhangs) such that the overhangs can hybridise with each other. The joining of the DNA fragments may be enzymatic, with a ligase enzyme, DNA ligase. However, a non-enzymatic ligation may also be used, as long as DNA fragments are joined, i.e. forming a covalent bond. Typically a phosphodiester bond between the hydroxyl and phosphate group of the separate strands is formed.

“Oligonucleotide primers” or “primers” in general, refer to strands of nucleotides which can prime the synthesis of DNA. DNA polymerase cannot synthesize DNA de novo without primers. A primer hybridises to the DNA, i.e. base pairs are formed. Nucleotides that can form base pairs, that are complementary to one another, are e.g. cytosine and guanine, thymine and adenine, adenine and uracil, guanine and uracil. The complementarity between the primer and the existing DNA strand does not have to be 100%, i.e. not all bases of a primer need to base pair with the existing DNA strand. From the 3′-end of a primer hybridised with the existing DNA strand, nucleotides are incorporated using the existing strand as a template (template directed DNA synthesis). We may refer to the synthetic oligonucleotide molecules which are used in an amplification reaction as “primers”.

“Primer based amplification” refers to a polynucleotide amplification reaction, namely, a population of polynucleotides that are replicated from one or more starting sequences, i.e. a primer. A suitable primer may have a sequence length of 15-30 nucleotides. Amplifying may refer to a variety of amplification reactions, including but not limited to polymerase chain reaction (PCR), linear polymerase reactions, nucleic acid sequence-based amplification, rolling circle amplification and the like.

“Sequencing” refers to determining the order of nucleotides (base sequences) in a nucleic acid sample, e.g. DNA or RNA. Many techniques are available such as Sanger sequencing and

“High throughput sequencing technologies, also referred to in the art as next generation sequencing, such as offered by Roche, Illumina and Applied Biosystems, or also referred to in the art as third generation sequencing, as described by David J Munroe & Timothy J R Harris in Nature Biotechnology 28, 426-428 (2010) and such as offered by Pacific Biosciences and Oxford Nanopore Technologies, may also be used. Such technologies allow from one sample DNA multiple sequence reads in a single run. For example, the number of sequence reads may range from several hundred up to billions of reads in a single run of a high through put sequence technology. High throughput sequencing technologies may be performed according to the manufacturer's instructions (as e.g. provided by Roche, IIlumina or Applied Biosystems). The technology may involve the preparation of DNA before carrying out a sequencing run. Such preparation may include ligation of adaptors to DNA. Adaptors may include identifier sequences to distinguish between samples. Depending on the size of DNA that is suitable or compatible with the high throughput sequencing technology used, the DNA that is to be sequenced may be subjected to a fragmenting step.

The term “contig” or “contiguous sequence” is used in connection with DNA sequence analysis, and refers to reassembled contiguous stretches of DNA derived from two or more DNA fragments having contiguous nucleotide sequences. Thus, a contig may be a set of overlapping DNA fragments that provides a (partial) contiguous sequence of a genomic region of interest. A contig may also be a set of DNA fragments that, when aligned to a reference sequence, may form a contiguous nucleotide sequence. For example, the term “contig” encompasses a series of (ligated) DNA fragment(s) of which the sequence has been determined which are ordered in such a way as to have a contiguous sequence (e.g. through overlap of (ligated) DNA fragment(s) and/or DNA fragment(s) positioned adjacently) with at least one of its neighbours. The linked or coupled (ligated) DNA fragment(s) may be ordered either manually or, preferably, using appropriate computer programs such as FPC, PHRAP, CAP3 etc., and may also be grouped into separate contigs.

“Size selection' according to the invention involves techniques with which particular size ranges of molecules, e.g. (ligated) DNA fragments or amplified (ligated) DNA fragments, are selected. Techniques that can be used are for instance gel electrophoresis, size exclusion, gel extraction chromatography, but are not limited thereto, as long as molecules with a particular size can be selected or excluded, such a technique will suffice.

With the term “aligning” and “alignment” is meant the comparison of two or more nucleotide sequence based on the presence of short or long stretches of identical or similar nucleotides. Methods and computer programs for alignment are well known in the art. One computer program which may be used or adapted for aligning is “Align 2”, authored by Genentech, Inc., which was filed with user documentation in the United States Copyright Office, Washington, D.C. 20559, on Dec. 10, 1991.

### FIGURES

FIG. 1A shows a schematic for generating DNA circles derived from genomic DNA wherein said DNA circles are generated from genomic DNA fragments. First DNA is provided, e.g. an isolated DNA. The DNA is depicted linearly with a black section indicating the target nucleotide sequence (viewpoint). Said isolated DNA is subsequently fragmented, e.g. by shearing. It is understood that isolating DNA inherently can have the result of fragmenting the DNA. The genomic DNA fragments that are generated consists of genomic DNA fragments containing a target nucleotide sequence and genomic DNA fragments not containing a target nucleotide sequence. The genomic DNA fragments are subsequently circularized to generate DNA circles. The DNA circles comprise DNA circles that contain a target nucleotide sequence (depicted as circles with a black section) and DNA circles not containing a target nucleotide sequence (depicted as circles without a black section).

FIG. 1B shows that the genomic DNA fragments that can be used to generate DNA circles, resulting e.g. from shearing, can have varying sizes of genomic sequences flanking the target nucleotide sequence (viewpoint). Hence, when on average a genomic DNA fragment size is obtained of about 50 kilobases, the random positioning of the target nucleotide sequence (viewpoint) in each 50 kilobases DNA fragment (each DNA fragment derived from a chromosome) causes that the genomic region of interest may cover up to about 100 kilobases.

FIG. 1C shows a schematic for generating DNA circles derived proximity ligation method for use in a method according to the invention. First, a sample DNA is provided. The sample DNA comprises crosslinked genomic DNA. The DNA is depicted linearly with a black section indicating the target nucleotide sequence. In its crosslinked state the DNA is fragmented, resulting in multiple crosslinked DNA fragments (indicated with A-H). One of the DNA fragments comprises the target nucleotide sequence (indicated with (D), viewpoint). The crosslinked DNA fragments are subsequently ligated. In this proximity ligation step, DNA fragments that are in each other's proximity are favourably ligated. After the ligation step the crosslinks are removed. This results in ligated DNA fragments that may already be in the form of DNA circles, and/or the ligated DNA fragments may be subjected to further processing steps to generate DNA circles. The DNA circles comprise DNA circles that comprise DNA fragments wherein one of the fragments comprises the target nucleotide sequence (depicted as circles with DNA fragments, one having the black section (D), viewpoint), and DNA circles that comprise DNA fragments that do not contain the target nucleotide sequence (depicted as circles without a black section).

FIG. 2 shows a schematic for providing DNA circles containing the target nucleotide sequence selectively with an origin of replication sequence and a selection gene. DNA circles comprise DNA circles that contain a target nucleotide sequence (which is the only DNA circle (A) depicted the black section indicating the target nucleotide sequence (viewpoint)) and DNA circles not containing a target nucleotide sequence. First the DNA circle containing the target nucleotide sequence is linearized by cutting at the target nucleotide sequence (B). This way, the DNA that originally was e.g. flanking the target nucleotide (derived from either the genomic DNA fragment or ligated DNA fragments) sequence is now positioned in between the two parts that originate from the target nucleotide sequence. This linearization can be carried out by highly selective digestion using e.g. Cas9/CRISPR or CRISPR-Cpf1 technology or the like, an inverse PCR reaction, an Achilles cleavage or a rare restriction site. Next, a DNA insert is provided that has ends that are compatible for ligation or recombination with the parts at the end of the linearized fragment that originate from the target nucleotide sequence. The DNA insert comprises an origin of replication and/or a selection marker.

(C) The compatibility between the DNA insert and the linearized DNA circle may be facilitated by two linker DNAs. The two linker DNAs can be designed such that one linker DNA has on one end sequence identity (e.g. 15 bp) with one given end of the target nucleotide sequence of the linearized DNA circle and on the other end sequence identity with one given end of the DNA insert (e.g. 15 bp) and the other linker DNA has on one end sequence identity (e.g. 15 bp) with the other end of the target nucleotide sequence of the linearized DNA circle and on the other end sequence identity with the other end of the DNA insert (e.g. 15 bp). By carrying out ligation independent cloning in a single step a DNA circle can be obtained that contains an origin of replication and/or a selection marker, wherein the origin of replication and/or selection marker are flanked by the sequence parts that originate from the target nucleotide sequence. This method has the advantage that one can use a universal DNA insert and one only needs to select a target nucleotide sequence and prepare linker DNA.

(D) The DNA insert may also comprise compatible ends (e.g. having 15 base pair identity with the target nucleotide sequence ends of the linear DNA circle) that allow ligation independent cloning or the like.

(E) The DNA insert may also be inserted using standard cloning techniques such as sticky end ligation. For example, when Achilles cleavage or an inverse PCR reaction provides for selectivity, or when a rare restriction enzyme is used. It is also possible that such a standard approach involves an intermediate step wherein first such a (rare) restriction enzyme site is introduced at the target nucleotide sequence similar to the introduction of a DNA insert as shown in (C) or (D).

The end result of the various possible strategies is a DNA circle derived from genomic DNA of which in the target nucleotide sequence an origin of replication and/or a selection marker has been introduced.

FIG. 3 shows a schematic of the introduction of two double stranded breaks in the target nucleotide in a DNA circle to provide for a linear double stranded DNA that is flanked by fragments (known ends, flanking the double stranded breaks) derived from the target nucleotide sequence. The target nucleotide sequence may be part of a single genomic DNA fragment comprised in a DNA circle that represents the genomic region of interest (shown in the schematic), but may also be part of a DNA fragment that is comprised in a DNA circle comprising ligated DNA fragments. The known ends can be used e.g. for recombination with a DNA insert comprising ends that are compatible with the known ends (compatible ends), the DNA insert comprising e.g. an origin of replication (Ori) and a selection gene (Amp, i.e. ampicillin resistance gene).

### DETAILED DESCRIPTION

In a first aspect of the invention, a selection and amplification method is provided of a genomic region of interest, wherein the genomic region of interest contains a (preferably single) target nucleotide sequence, wherein the method comprises:
providing DNA circles derived from genomic DNA, wherein said DNA circles comprise DNA circles containing the target nucleotide sequence;
editing the target nucleotide sequence such that the DNA circles containing the target nucleotide sequence are provided with an origin of replication sequence and a selection gene;
transferring the DNA circles comprising the DNA circles containing said origin of replication sequence and said selection gene into a host cell; and
selectively culturing host cells comprising DNA circles containing said origin of replication sequence and said selection gene.

The method of the invention relates to a method for selection and amplification of a genomic region of interest, wherein said genomic region of interest contains a target nucleotide sequence. Hence, first of all some DNA sequence information is required regarding the target nucleotide sequence. In order to carry out the methods of the invention a genomic region of interest needs to be provided, e.g. a genomic region of interest that contains a target nucleotide sequence. For example, in case the genomic region of interest is of human origin, a complete reference genome may be provided. Because a complete reference genome is available, a target nucleotide sequence can easily be provided that is contained in the genomic region of interest, e.g. a gene of interest. In case no complete reference genome is available, a sequence of a genomic region of interest can be provided, e.g. a reference gene sequence. In case no genomic region of interest can be provided, the minimal sequence information required is a target nucleotide sequence, e.g. a DNA sequence identified as a molecular marker that is associated e.g. with a disease or a trait. A target nucleotide sequence may also be contained in a transgene which may be the only sequence information available and that has been integrated in a host genome. Hence, the target nucleotide sequence may be the only sequence information available by which a genomic region of interest that extends beyond the target nucleotide sequence is identified.

Whatever sequence information is provided, based on the sequence information available a target nucleotide sequence is selected. The target nucleotide sequence needs to allow for selective opening of the DNA circles containing the target nucleotide sequence. Selective in this respect refers to the opening of DNA circles containing the target nucleotide sequence, as opposed to DNA circles not containing the target nucleotide sequence. which may require the target nucleotide sequence to be at least 16-23 nucleotide sequence in length if opening is mediated by sequence-specific CRISPR/Cas9 or CRISPR-Cpf1 nuclease digestion, or at least ten nucleotides in length when e.g. a rare cutting restriction enzyme is used. The target nucleotide sequence subsequently also needs to allow for inserting a DNA insert such as a DNA insert carrying an origin of replication and/or a selection gene. The editing of the target nucleotide sequence comprises the opening of the DNA circle. The opening of the DNA circle comprises cutting within the target nucleotide sequence, or directly adjacent the target nucleotide sequence or near, i.e. at a known distance (known number of nucleotides, typically from 2-25 nucleotides away in case of a type IIs rare cutting restriction enzyme) of the target nucleotide sequence. Whether the opening of the target nucleotide sequence is within, adjacent or near depends on the means for opening the circle, such as for instance the use of type IIs restriction endonucleases. The editing further comprises the introduction of a DNA insert at the opening of the DNA circle.

This may require the target nucleotide sequence to be at least 10 nucleotides in length when insertion is carried out by ligation into a rare cutting restriction enzyme recognition site, or at least 2×15 nucleotides if insertion is carried out by ligation independent cloning. The length of the target nucleotide sequence is therefore at least 10 nucleotides, but is preferably at least 30 nucleotides. Preferably, the target nucleotide sequence that is selected is unique (not necessarily taking into account ploidy of the genome) within the context of the genomic DNA. For example, the human genome has about 3 billion base pairs. Hence, statistically, a randomly selected nucleotide sequence of 30 base pairs in length provides for a unique target nucleotide sequence in the genome (4³⁰>>3×10⁹). Furthermore, the target nucleotide sequence that is selected also depends on the technology used for amplification. For host cell amplification in accordance with the invention, the target nucleotide sequence must be selected such that it allows the insert of an origin of replication and/or a selection marker. This depends on the technology applied such as described below. When for example a targeted proximity ligation amplification method is used, i.e. DNA circles are formed wherein each circle comprising several DNA fragments that are derived from the genome, the target nucleotide sequence is selected such that it is accommodated in a DNA fragment derived from the genomic region of interest.

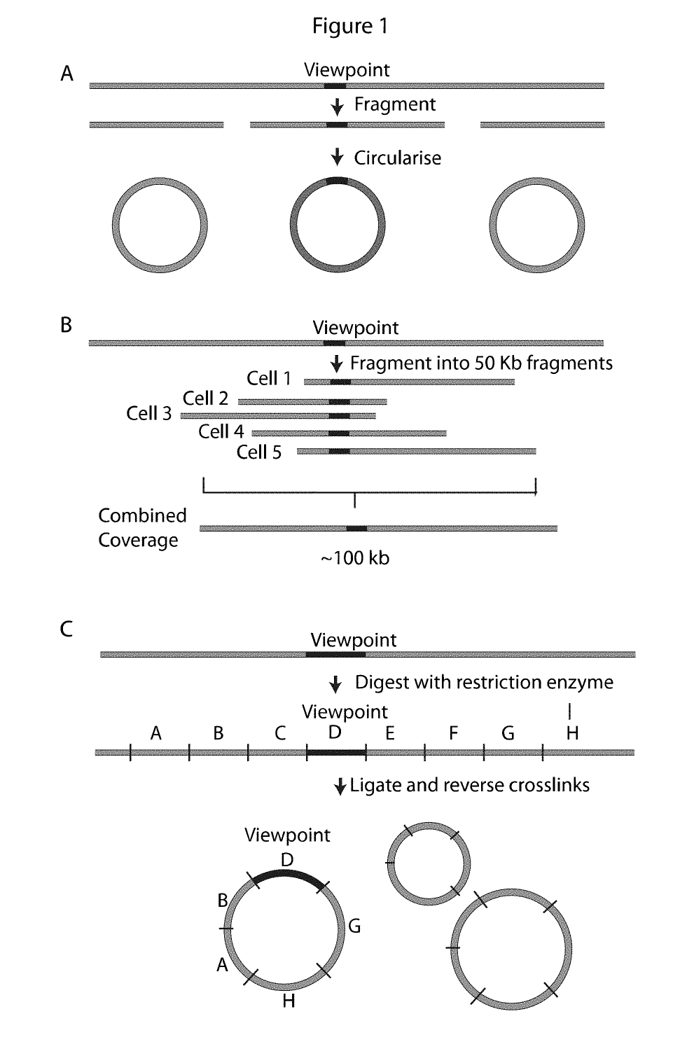

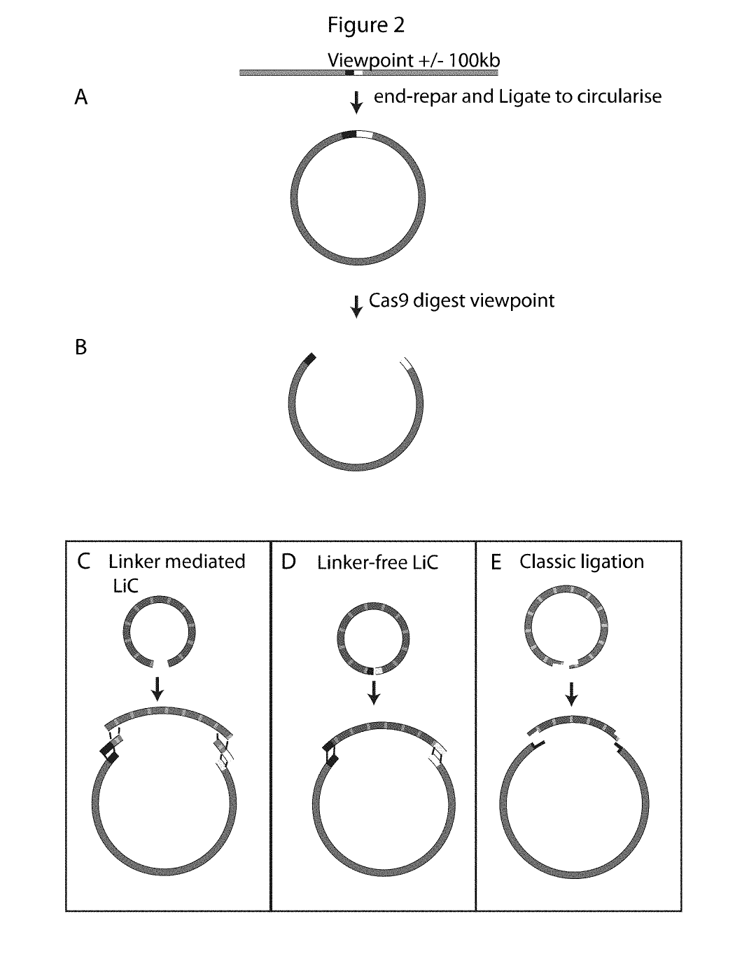

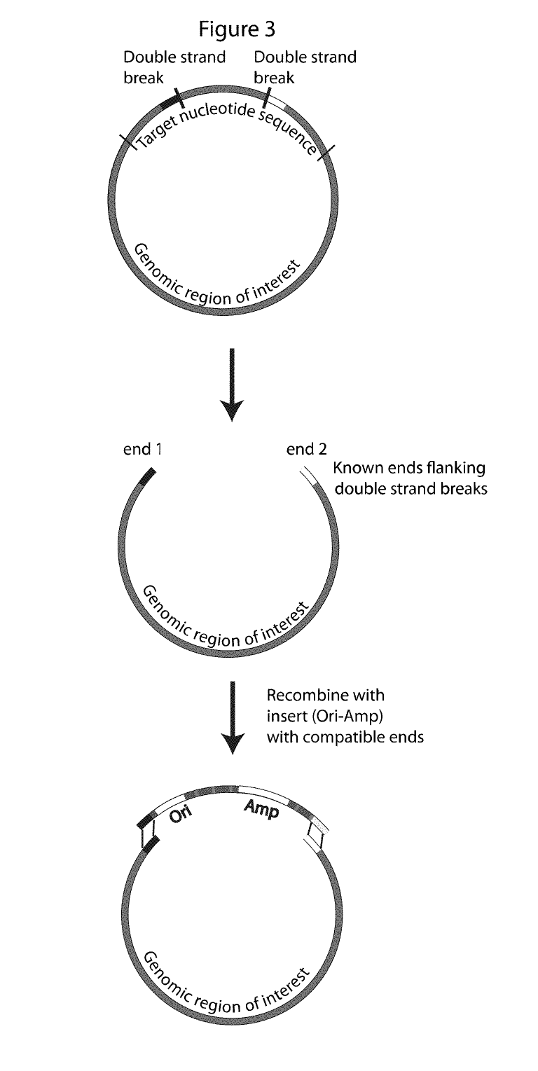

The first step of the method is to provide for DNA circles derived from genomic DNA, wherein said DNA circles comprise DNA circles containing the target nucleotide sequence. In one embodiment, the step of providing said DNA circles derived from genomic DNA comprises the steps of:
providing a sample of genomic DNA;
fragmenting the genomic DNA to provide genomic DNA fragments, wherein said genomic DNA fragments comprise genomic DNA fragments containing the target nucleotide sequence;
circularizing the genomic DNA fragments to obtain DNA circles derived from genomic DNA, wherein said DNA circles comprise DNA circles containing the target nucleotide sequence.
   In this embodiment providing a sample of genomic DNA relates to any basic technique involving DNA isolation, e.g. such as isolated from a tissue sample or other sample from an organism that comprises genomic DNA, and providing the genomic DNA that in a suitable condition (e.g. a suitable buffer) for the subsequent fragmenting and circularization steps. The size of the genomic DNA fragments is preferably in the range of 10 kb-300 kb, preferably in the range of 20 kb-100 kb, more preferably in the range of 30-60 kb. It is understood that the method of invention yields a genomic region of interest that flanks on both sides the target nucleotide sequence (see FIG. 1B). Since in standard genomic DNA isolation procedures DNA is randomly fragmented, each DNA fragment of a given length carrying the target nucleotide sequence will have the target nucleotide sequence placed at another position. Therefore, when e.g. the genomic DNA fragments are about 30 kb in length, the part of the genomic region of interest that can be analysed may extend to about 60 kb. When e.g. the genomic DNA fragments are up to 60 kb in length, the part of the genomic region of interest that can be analysed may extend to about 120 kb. Hence preferably, the target nucleotide sequence may be selected such that is in the centre range of the genomic region of interest. The DNA circles that are formed from the genomic DNA fragments can be made using standard procedures, and may include enzymatically blunting and/or repairing and/or polishing genomic DNA fragments to render these compatible for ligation. Also, appropriate conditions may be selected to promote circularization, such as lower temperatures during ligation, extended ligation periods and/or diluted DNA concentrations.

As said, the first step of the method is to provide for DNA circles derived from genomic DNA, wherein said DNA circles comprise DNA circles containing the target nucleotide sequence. In another embodiment, the step of providing said DNA circles derived from genomic DNA comprises the steps of:
providing a sample of crosslinked DNA;
fragmenting the crosslinked DNA to provide for crosslinked DNA fragments, wherein said crosslinked DNA fragments comprise crosslinked DNA fragments containing the target nucleotide sequence;
ligating the crosslinked DNA fragments to provide for ligated crosslinked DNA fragments;
reversing the crosslinking to provide for ligated DNA fragments;
generating DNA circles derived from genomic DNA from the ligated DNA fragments.
   This embodiment relates to targeted proximity ligation amplification methods that comprise all the steps required to provide for DNA circles comprising ligated DNA fragments (such as e.g. described in WO2012005595, WO2014129894, WO 2007/004057 and WO 2008/08845 which are included herein by reference). Such methods may be advantageous when e.g. a sample DNA is already composed of crosslinked fragmented DNA, as is the case for formalin fixed paraffin embedded samples (FFPE). DNA fragments comprised in DNA circles according to the invention preferably have a size in the range of 200-500 bp, and the DNA circles comprising said DNA fragments preferably have a size in the range of 1-20 Kb. The size of the DNA fragments is preferably smaller than the size of the DNA circles and such that several DNA fragments are comprised in a DNA circle. The size of the genomic regions of interest that can be covered by targeted proximity ligation amplification methods is up to 2 Mb around a given target nucleotide sequence. Such targeted proximity ligation amplification methods comprise at least the step of fragmenting DNA (such as crosslinked DNA) to obtain crosslinked DNA fragments and wherein said obtained crosslinked DNA fragments are ligated via proximity ligation to obtain DNA circles. The ligated crosslinked DNA fragments subsequently have the crosslinks reversed. Uncircularized ligated DNA fragments can subsequently be circularized to form DNA circles.

Hence, in both embodiments as described above, the DNA circles derived from genomic DNA that are formed either comprise a target nucleotide sequence, or do not comprise a target nucleotide sequence. The method of the invention requires DNA circles in order to obtain a suitable double stranded DNA that can be propagated in a host cell, such as e.g. bacteria or yeast. In order to allow propagation in a host cells, the DNA circles with the target nucleotide sequence are provided with an origin of replication and a selection gene. DNA circles not having the target nucleotide sequence are not provided with an origin of replication and a selection gene. DNA circles comprising the target nucleotide, as derived from ligated DNA fragments or genomic DNA fragments, are modified such that at the target nucleotide sequence an origin of replication sequence and a selection gene sequence are inserted (or, at least one of these). Hence, the target nucleotide sequence may not necessarily be in the sequence context such as it was originally present in the genomic DNA after the DNA circle comprising an origin of replication and/or a selection gene has been formed. In any case, in accordance with the invention, at any stage of all the steps subsequent to the formation of DNA circles the insertion of an origin of replication sequence and/or a selection gene sequence at the target nucleotide sequence is contemplated, as long as the result is that DNA circles are formed, of which the DNA circles with the target nucleotide sequence have an insertion at the target nucleotide sequence such that said circles have a selection gene sequence and an origin of replication sequence.

Said DNA circles are next transferred into a host cell. Suitable host cells that can be contemplated in the methods of the invention are preferably bacterial cells, or alternatively yeast. The said origin of replication is selected such that the DNA circle can be propagated in the host cell. For a bacterial host cell a suitable bacterial origin of replication is used, for a yeast cell a yeast host cell origin of replication is used. Preferably an origin of replication is used with a low copy number in the host cell. The selection gene allows to select for transformed host cells that carry the DNA circles with the target nucleotide sequence. The transformed host cells are next cultured in a suitable medium to allow the transformed host cell to express the selection gene. When for example the selection gene is an antibiotic resistance gene, said transformed cells can be cultured in a suitable medium that provides for all the required nutrients of the host cell and an appropriate antibiotic such that only the transformed host cells with the DNA circles with the origin of replication and antibiotic resistance gene will grow and transformed host cells not carrying the said origin of replication and said antibiotic resistance gene will not grow. This way, only DNA circles that carry the target nucleotide sequence are selected and amplified in the host cell.

The DNA circles that are selected and amplified in the host cell, i.e. DNA circles that have the origin of replication and a selection gene, can next be isolated and subjected to standard methods such as used in high throughput sequencing methods or microarrays, for example as also used in the prior art in TLA, 4C and 3C methods.

Hence, as outlined above, TPLA methods in accordance with the current invention apply an amplification step using host cell amplification instead of PCR amplification, wherein at the target nucleotide sequence an origin of replication and/or an antibiotic resistance gene is inserted in order to allow host cell amplification of DNA circles with the target nucleotide sequence because these comprise an origin of replication and an antibiotic resistance gene.

Genomic regions of interest may thus be analysed according to the invention and compared with a reference, or different genomic regions of interest may be analysed and compared with each other. For example, sample DNAs may be provided from a patient being suspected of having breast cancer, a biopsy may be obtained from the suspected tumour. Another biopsy may be obtained from non-diseased tissue. From both tissue biopsies may be subjected to the methods of the invention. By determining the genomic region of interest sequence according to the invention and comparing the genomic region sequences of the different biopsies with each other and/or with a reference gene sequence, genetic mutations may be found that will assist in diagnosing the patient and/or determining treatment of the patient and/or predicting prognosis of disease progression.

Hence, embodiments according to the invention for selecting and amplifying genomic regions of interest comprising a target nucleotide sequence either comprise a step of providing a sample of crosslinked DNA or a step of providing a sample of genomic DNA.

In said embodiments comprising a step of providing a sample of genomic DNA, the said genomic DNA is fragmented, e.g. by shearing. In any isolation method of genomic DNA the genomic DNA is fragmented e.g. due to resuspending DNA with pipetting and mechanical shearing and removal of packaging proteins, usually into large fragments that are 50 kb or larger in size. Optional further fragmentation methods include sonication, mechanical shearing or enzymatic digestion. Random fragmenting during standard genomic DNA isolation procedures can occur as a result of aforementioned reasons such as mechanical shearing, pipetting, protein removal and other forces applied during DNA purification results in the fragmenting of DNA at random sites. These can be either blunt ended, or can have 3′- or 5′- overhangs. The DNA may be repaired (enzymatically), filling in possible 3′- or 5′- overhangs, such that DNA fragments are obtained which have blunt ends. Alternatively, the overhangs may also be made blunt ended by removing overhanging nucleotides, using e.g. exonucleases. When filling-in the ends, optionally biotinyated nucleotides may be incorporated, such that end-repaired DNA fragments carry a biotin moiety that enables separating the end-repaired DNA fragments from the not-end-repaired DNA fragments. Such separation of biotinylated DNA fragments from non-biotinylated DNA fragments may be carried out prior to, or after the subsequent ligation step. End-repair of the DNA fragments enables intra-molecular ligation of the fragments in the subsequent step, such that the one end of a given fragment ligates to its other end. The result hereof is that each fragment is turned into an individual DNA circle, or that some of the fragments are turned into individual DNA circles.

To promote intramolecular ligation (i.e. to promote that ligations between the ends of the same fragment are preferred over ligations between ends of two different fragments) ligation can be carried under diluted DNA conditions such that the DNA concentration is less than e.g. 100 ng/μl, or preferably less than 10 ng/μl. As another means to promote intramolecular ligation DNA fragments may be first attached to a solid matrix such as SPRI (Solid Phase Reversible Immobilization) beads, AMPure beads (e.g. NEB, Agilent, Beckman Coulter), Dynabeads (Thermofisher), DNA immobilized glass beads, etc., followed by ligation. Further, to favour intramolecular ligation fragments may be first sheared to create fragments with a size of 50 kb, 30 kb or 20 kb. Also, the blunted DNA ends of the fragments may be enzymatically provided with an adenosine at the 3′ end (known in the field as ‘A-tailing’) to promote ligation. In such scenario a double stranded linker or double stranded backbone with a 3′ thymidine (3′T) overhang on both ends can be added prior to ligation to stabilize hybridization between the complementary A and T and promote ligation between the linker or backbone carrying the 3′T overhangs and the DNA fragment carrying the 3′A overhangs. It is understood that such linker or backbone can carry a biotin moiety that enables separating the DNA fragments ligated to the linker or backbone from DAN fragments not ligated to the linker or backbone. It is also understood that the various methods for promoting ligation and/or promoting intramolecular ligation can also be combined.

In embodiments comprising a step of providing a sample of crosslinked DNA the crosslinked DNA comprises genomic DNA as it is present e.g. in a sample DNA, largely maintaining the three dimensional architecture of the genomic DNA. A standard crosslinking agent that may be used is formaldehyde. By fragmenting the sample of crosslinked DNA, the DNA fragments that originate from a genomic region of interest remain in proximity of each other because they are crosslinked. Hence, the fragmenting step results in the introduction of double stranded breaks in the DNA while the crosslinks remain. When these crosslinked DNA fragments are subsequently ligated, DNA fragments of the genomic region of interest, which are in the proximity of each other due to the crosslinks, are ligated. This type of ligation is also referred to as proximity ligation. Crosslinked DNA fragments comprising the target nucleotide sequence may ligate with DNA fragments within a large linear distance on sequence level.

The fragmenting step of crosslinked DNA may comprise sonication, and may be followed by enzymatic DNA end repair. Sonication results in the fragmenting of DNA at random sites, which can be either blunt ended, or can have 3′- or 5′- overhangs, as these DNA breakage points occur randomly, the DNA may be repaired (enzymatically), filling in possible 3′- or 5′- overhangs, such that DNA fragments are obtained which have blunt ends that allow ligation of the fragments to adaptors or each other in the subsequent step c). Alternatively, the overhangs may also be made blunt ended by removing overhanging nucleotides, using e.g. exonucleases. The fragmenting step of crosslinked DNA may also comprise fragmenting with a restriction enzyme, or combinations thereof. Fragmenting with a restriction enzyme is advantageous as it may allow more control of the average fragment size. Furthermore, the fragments that are formed will have compatible overhangs or blunt ends that allow ligation of the fragments in the subsequent step without requiring further modification. When filling-in the ends, optionally also after restriction enzyme digestion, optionally biotinyated nucleotides may be incorporated, such that DNA fragments carry a biotin moiety enabling separating the end-repaired and digested DNA fragments from not-end-repaired and non-digested DNA fragments.

The fragments are next ligated. In case the DNA fragments were obtained via enzymatic restriction, the recognition site of the restriction enzyme is known, which makes it possible to identify the fragments, as remains of or reconstituted restriction enzyme recognition sites may indicate the separation between different DNA fragments. In case the DNA fragments were obtained via random fragmentation, such as sonification and subsequent enzymatic DNA end repair, it may be more difficult to distinguish one fragment from another.

Next, the crosslinking is reversed which results in a pool of ligated DNA fragments that comprise two or more fragments. A subpopulation of the pool of ligated DNA fragments comprises a DNA fragment which comprises the target nucleotide sequence. By reversing the crosslinking, the structural/spatial fixation of the DNA is released and the DNA sequence becomes available for subsequent steps, as crosslinked DNA may not be a suitable substrate for such steps.

The said ligated DNA fragments may be already in the form of DNA circles, the said ligated DNA fragments may also be in the form of linear DNA, or a mixture of linear DNA and DNA circles. Proximity ligation may result e.g. in some DNA circles, e.g. about 10% of ligated DNA fragments may be circular, and a further ligation may yield more DNA circles. Hence, a further ligation step may be carried out to provide for DNA circles. Prior to the further ligation step, an option may be to introduce a further fragmenting step in order to obtain DNA circles of a preferred size. In any case, any further fragmenting step results in linear DNA fragments comprising multiple DNA fragments that originate from the first fragmenting step. These are then ligated to form DNA circles.

As said, embodiments according to the invention for selecting and amplifying genomic regions of interest comprising a target nucleotide sequence either comprise a step of providing a sample of crosslinked DNA or a step of providing a sample of genomic DNA. In both strategies DNA circles are generated derived from genomic DNA. After ligation, DNA circles may be present in a mix with linear fragments. To remove the linear fragments, exonuclease digestion may be carried out.

Next, said DNA circles with the target nucleotide sequence are provided with an origin of replication sequence and a selection gene. It is understood that when said DNA circles containing the target nucleotide sequence are (or have been) provided with an origin of replication and a selection gene, this means that DNA circles not containing the target nucleotide sequence have not been provided with an origin of replication and a selection gene.

Suitable selection genes according to the invention are e.g. antibiotic resistance gene, but may also include genes expressing fluorescent genes or the like that may be suitable e.g. for FACS sorting. The selection gene may be selected from the group consisting of an antibiotic resistance gene, a pigment gene, a fluorescent gene, and a host cell surface protein gene. Such selection genes that are provided can be expressed in the host cell and allow for selecting host cells that have been provided therewith while host cells not provided therewith are not selected or do not survive (such as with antibiotics). The origin of replication that is provided allows the host cell to propagate the DNA circles. Hence, the DNA circles that have an origin of replication and a selection gene are basically plasmids that contain either the ligated DNA fragments or genomic DNA fragment that represent the genomic region of interest.

When the host cells are next cultured, e.g. in an appropriate medium containing all the necessary nutrients and, e.g. in case of an antibiotic resistance gene as a selection gene an appropriate antibiotic, the host cells that contain the origin of replication and selection gene multiply. In case e.g. a fluorescent gene is provided as a selection gene, first host cells containing said selection gene are first allowed to express said gene after which the host cells with the fluorescent gene can be selected for by fluorescent activated cell sorting. Hence, host cells comprising DNA circles with said origin of replication sequence and said selection gene are selectively cultured. After culturing, the DNA circles with the origin of replication and selection gene are isolated. After the isolation step the DNA circles can be subjected to high throughput sequencing methods or to microarray analysis methods such that sequence information of the genomic region of interest is obtained. The host cells that are provided with the DNA circles containing said origin of replication sequence and said selection gene may be cultured in a large pool, i.e. a single culture comprising all DNA circles derived from different cells that contain a target nucleotide sequence. Alternatively, the said host cells may be cultured as cell clones (e.g. on a culture plate). When cell clones are cultured the advantage is that it is known that each clone is derived from one allele (with the exception of double transformed cells). The advantage of clones is that independent of the sequencing method one knows that sequences derived from a clone represent an allele.

In a further embodiment, the DNA circles that are generated can have an origin of replication sequence and a selection gene inserted at the target nucleotide sequence. DNA circles not having the target nucleotide sequence are not provided with the selection gene inserted at the target nucleotide sequence. Hence, DNA circles that are generated have an origin of replication sequence and a selection gene selectively inserted at the target nucleotide sequence. Alternatively, the DNA circles that are generated can have a selection gene inserted and an origin of replication sequence is inserted at the target nucleotide sequence. DNA circles not having the target nucleotide sequence thus only carry the selection gene and only the DNA circles having the target nucleotide sequence carry the selection gene and the origin of replication. Conversely, the DNA circles that are generated can have an origin of replication inserted, and have a selection gene inserted at the target nucleotide sequence. Only the DNA circles with the target nucleotide sequence are provided with an origin of replication and a selection gene, while DNA circles not having the target nucleotide sequence are only provided with an origin of replication. Hence, the DNA circles with the target nucleotide sequence that are generated according to the invention are selectively provided with both an origin of replication sequence and a selection gene. For example, when the blunted DNA ends of the fragments are enzymatically provided with an adenosine at the 3′ end (known in the field as ‘A-tailing’) and a double stranded backbone with a 3′ thymidine (3′T) overhang on both ends is provided to stabilize hybridization between the complementary A and T and promote ligation between the backbone carrying the 3′T overhangs and the DNA fragment carrying the 3′A overhangs, this backbone may contain an origin of replication and/or a selection gene.

The step of selectively inserting an origin of replication sequence and/or a selection gene at the target nucleotide can comprise the steps of:
providing a linear DNA molecule derived from said DNA circles containing the target nucleotide sequence, having fragment ends, each fragment end derived from the target nucleotide sequence;
providing a DNA insert comprising the origin of replication sequence and/or a selection gene, wherein said DNA insert is a linear DNA molecule having fragment ends, each fragment end being compatible with the said fragment ends derived from the target nucleotide sequence;
allowing the linear DNA molecule and DNA insert to form a DNA circle containing an origin of replication sequence and/or a selection gene.

The step of selectively inserting an origin of replication sequence and a selection gene at the target nucleotide sequence can comprise the steps of;
linearizing the DNA circles with the target nucleotide sequence to provide a linear DNA molecule having fragment ends, each fragment end derived from the target nucleotide sequence;
providing a DNA insert comprising the origin of replication sequence and a selection gene, wherein said DNA insert is a linear DNA molecule having fragment ends, each fragment end being compatible with the said fragment ends derived from the target nucleotide sequence;
allowing the linear DNA molecule and DNA insert to form a DNA circle containing an origin of replication sequence and a selection gene.

In another embodiment, the DNA insert may have compatible restriction sites positioned flanking the origin of replication sequence and/or selection gene such that the origin of replication sequence and/or selection gene may be removed after selection and amplification by restriction and subsequent ligation.

The DNA circles that carry the target nucleotide sequence have either first introduced at least a double stranded break at the target nucleotide sequence, or are subjected to an inverse PCR reaction of DNA circles comprising the target nucleotide sequence in order to provide for a linear double stranded DNA molecule having at each end part of the target nucleotide sequence, i.e. fragment ends that originate from the target nucleotide sequence. When the DNA circle derived from genomic DNA is based on circularized genomic DNA fragment, the DNA circle containing the target nucleotide sequence may be linearized by e.g. cutting at the target nucleotide sequence (e.g. once or twice), and the DNA that originally was flanking the target nucleotide sequence may now be positioned in between two parts that originate from the target nucleotide sequence. When the DNA circle derived from genomic DNA is based on circularized ligated DNA fragments, the DNA circle containing the target nucleotide sequence may be linearized e.g. by cutting at the target nucleotide sequence (e.g. once or twice), and the DNA fragment that originally contained the target nucleotide sequence may now be divided in two parts (or more), each part positioned at the end of the linearized DNA circle and having DNA fragments not containing the target nucleotide sequence in between.

It is understood that the double stranded break that is introduced at the target nucleotide sequence may be the result of one double stranded DNA break in the DNA circle. It is also understood that the target nucleotide sequence may have two double stranded DNA breaks in the target nucleotide sequence or two nicks in the target nucleotide sequence (see e.g. Ran FA, et al., Cell. 2013 Sep. 12; 154(6):1380-9) to obtain the linear double stranded DNA molecule having at each end part of the target nucleotide sequence, i.e. fragment ends that originate from the target nucleotide sequence (see e.g. FIG. 3). For example, when the target nucleotide sequence is present in a DNA circle derived from multiple ligated DNA fragments, the target nucleotide sequence may be represented by a DNA fragment. When this DNA fragment (the target nucleotide sequence) is e.g. 2 kb (kilo base pairs) in size, fragmenting the DNA circle once at the centre of this 2 kb DNA fragment results in a linear double stranded DNA molecule with at each end a fragment derived from the target nucleotide sequence of about 1 kb in size. When this DNA circle would be fragmented two times (or more) such that a midsection of the DNA fragment is removed of about 1.6 kb, the result is a linear double stranded DNA molecule with at each end a fragment derived from the target nucleotide sequence of about 0.2 kb in size. For TLA approaches the (at least) two double stranded DNA breaks introduced in the target nucleotide sequence may preferably be at most 4 kb, more preferably at most 2 kb apart (relative to the reference nucleotide sequence of the target nucleotide sequence as it is originally present in the genome). The maximum distance between the two double stranded DNA breaks is determined by the size of the DNA fragments that are generated when the DNA is fragmented in the crosslinked state, or by the size of the fragments that are generated when the DNA is fragmented after reversing the crosslinking. Likewise, for DNA circles that are derived from single genomic DNA fragments, the DNA circle may also have introduced at least one double stranded DNA break at the target nucleotide sequence. For DNA circles the target nucleotide sequence may be larger as compared to target nucleotide sequence used in TLA like approaches because there is no limit due to the size of DNA fragments. Hence, the (at least) two double stranded DNA breaks introduced in the target nucleotide sequence may for DNA circles having a single genomic DNA fragment be at most 10 kb apart (relative to the reference nucleotide sequence of the target nucleotide sequence as it is originally present in the genome). Preferably, for DNA circles in general the (at least) two double stranded DNA breaks introduced in the target nucleotide sequence may be about 1 kb apart, preferably about 500 bp (base pairs), preferably about 400 bp, about 300 bp, most preferably about 200 bp apart. The size ranges in between at least two double stranded DNA breaks as described above may also apply to inverse PCR reactions for the spacing of the primer binding sites. When primer binding sites or two double stranded DNA breaks or two nicks in the target nucleotide sequence are spaced apart it is understood that the intervening sequence information is lost. If genomic fragments comprised of ligated DNA fragments are contained in DNA circles, the use of two breaks and resulting loss of intervening target nucleotide sequence may be advantageous to avoid excess amplification of the of intervening target nucleotide sequence. The use of two breaks may also be advantageous for the selection of two unique sequence ends that facilitate the introduction of a DNA insert carrying an origin of replication and/or selection gene. Whatever method is used to generate the linear double stranded DNA molecule from a DNA circle having a target nucleotide sequence, as long as a linear double stranded DNA molecule having at each end part of the target nucleotide sequence is generated that allows the introduction of the DNA insert carrying the origin of replication and/or selection gene, such a method is contemplated in the invention. Hence, with regard to the target nucleotide sequence, the minimal sequence information that needs to be known relates to the sequence information that is needed to generate the said linear DNA molecule and that allows the introduction of the DNA insert carrying the origin of replication and/or selection gene.

Next, a DNA insert is provided that comprises an origin of replication sequence and/or a selection gene, wherein said DNA insert is a linear DNA molecule having insert ends, each insert end being compatible with the said fragment ends derived from the target nucleotide sequence. Compatible ends according to the invention means that the ends have compatible overhangs, and/or have sequence identity to allow for the linear DNA molecule and DNA insert to form a DNA circle. For example, a restriction enzyme can result in specific overhangs that allow the DNA insert and linear DNA molecule to ligate using e.g. standard cloning techniques. Furthermore, homologous recombination techniques or ligation independent cloning techniques or the like, that require sequence identity between the fragment ends of the linear DNA molecule and the DNA insert to generate a DNA circle. The sequence identity for the latter techniques require at least 15 base pairs of substantial sequence identity, preferably the sequence identity for the at least 15 base pairs is 100%.

As said, the fragment ends of the linear DNA molecule can be generated by introducing a double stranded break at the target nucleotide sequence. Such methods can involve e.g. the use of a restriction enzyme. For example, if the method generates DNA circles of a certain size, an appropriate restriction enzyme can be selected for that restricts the target nucleotide sequence such that DNA circles on average will be restricted only once. For example, if DNA circles have an average size of about 4 kB, and the target nucleotide sequence comprises a 6 base pair recognition site (theoretically present once in every 4096 basepairs), restriction with said enzyme results in about 1 restriction per DNA circle on average. When larger DNA circles are preferred, a larger restriction site may be selected. However, there is not always an appropriate restriction site available for a given target nucleotide sequence or a given genomic region of interest that allows for only one restriction per DNA circle on average.

Hence, the double stranded DNA break can also be introduced by using an Achilles Cleavage method, such as described i.a. by Koob et al. NAR, Vol. 20 No. 21, 5831-5836, 1992. In such a method, use is made of restriction enzymes that do not restrict a methylated recognition site. The target nucleotide sequence is protected from enzyme recognition, e.g. by using a blocker probe that hybridizes to the target nucleotide sequence, or by using an enzyme that binds to the target nucleotide sequence. When next the DNA is methylated, the enzyme recognition site is not methylated whereas the recognition site elsewhere is. This way, the enzyme recognition site that can be digested by the methylation sensitive restriction enzyme is in the target nucleotide sequence. In any case, these methods all rely on the use of restriction enzymes. Double stranded DNA breaks may also be introduced by using sequence specific genome editing nucleases, such as CRISPR/Cas9 (as e.g. available from catalogue #M0386L, manual M0386, New England Biolabs, Ipswich, UK) or CRISPR-Cpf1 (as described e.g. in Zetche et al. Cell, September 2015) which can be guided to or designed such that they recognize a specific sequence in the target nucleotide sequence, a sequence that is e.g. unique or occurs rarely in the genome (e.g. a recognition site of 15 nucleotides occurs statistically 1 in every billion base pairs).

The linear DNA molecule having at each end part of the target nucleotide sequence, i.e. fragment ends that originate from the target nucleotide sequence, can also be provided by an inverse PCR reaction of DNA circles comprising the target nucleotide sequence. Such an approach is in fact similar to the inverse PCR reactions typically carried out in TPLA methods or the like such as described in the prior art. Inverse primers, each having a size ranging e.g. from 15-30 nucleotides, provide for a selective amplification of only DNA circles that have a DNA fragment comprising (or consisting of) the target nucleotide sequence or of genomic DNA fragments comprising the target nucleotide sequence. Preferably the number of cycles in the PCR reaction ranges from 2-20. 2 cycles is the minimum as it allows the formation of a double stranded linear DNA.

In a further embodiment, the step of selectively inserting an origin of replication sequence and/or a selection gene at the target nucleotide comprises a method selected from the group consisting of:
homologous recombination;
restriction and ligation;
ligation independent cloning.

This step can correspond with the step of allowing the linear DNA molecule and DNA insert to form a DNA circle. For example, the compatible ends of the linear DNA molecule and the DNA insert may be designed and/or selected such that it allows homologous recombination, restriction and ligation or ligation independent cloning. These technologies are commercially available, e.g.:
In-Fusion HD kit (catalogue #638909-638911, 638916-638920), In-Fusion HD Cloning Kit User Manual 011614, Clontech, Mountain View, USA,
PPY-bacterial extract mediated recombination (as described in Zhang, Y. et al., Methods Mol Biol, 2014. 1116: p. 235-44)
Gibson Assembly Cloning (catalogue #E5510S), described in protocol E5510, New England Biolabs, Ipswich, UK),
Gene-Art seamless cloning (catalogue #A13288, A14603, or A13289), described in GENEART Seamless Cloning and Assembly Kit manual MAN0003222, Thermo Fisher Scientific, Waltham, USA
Gateway cloning (catalogue #12535-029 and 12535-037), as described in User Guide: Gateway Technology 25-0749, Thermo Fisher Scientific, Waltham, USA,
RecA mediated recombination as described in Li, M. Z. and S. J. Elledge, Nat Methods, 2007. 4(3): p. 251-6, (catalogue #M0249S or M0249 L combined with M0203S or M0203L) New England Biolabs, Ipswich, UK

It is the objective in the invention to provide for DNA circles that have the target nucleotide sequence to have inserted an origin of replication sequence and a selection gene at the target nucleotide, such that DNA circles not having the target nucleotide sequence do not have the origin of replication sequence and the selection gene. This can be accomplished as exemplified above, by introducing e.g. a double stranded break only in the target nucleotide (e.g. by using Achilles Cleavage, by using sequence specific genomic editing nuclease), by using an inverse PCR reaction, by using homologous recombination, or by using ligation independent cloning. Such steps, or the like, because they can depend on DNA sequences of about 15 nucleotide or longer allow to selectively insert an origin of replication sequence and/or a selection gene at the target nucleotide sequence.

In a further embodiment, the step of selectively inserting an origin of replication sequence and/or a selection gene at the target nucleotide sequence comprises a method that involves any of the methods described above but using a primary, facilitator, DNA insert that comprises the recognition sequence of an extremely rare cutting restriction endonuclease such as I-Scel. The selective insertion of an I-Scel recognition sequence into DNA circles containing the target nucleotide sequence facilitates, by I-Scel digestion and ligation, the subsequent insertion of the secondary, critical, DNA insert comprising an origin of replication and/or a selection gene and having I-Scel compatible DNA overhangs, selectively into the DNA circles containing the target nucleotide sequence. Further selectivity can for example also be provided for by using a capture step. For example, genomic DNA fragments, ligated DNA fragments or DNA circles comprising the target nucleotide sequence can be captured with a probe capable of hybridising to the target nucleotide sequence and separated from ligated DNA fragments or DNA circles not comprising the target nucleotide sequence. Such capture step may be performed as a first means of enrichment of genomic fragments, ligated DNA fragments or DNA circles comprising the target nucleotide sequence, to facilitate subsequent introduction of the origin of replication and/or selection gene into the target nucleotide sequence and/or to facilitate successful transformation of the host cell with DNA circles carrying the origin of replication and/or selection gene inserted into the target nucleotide sequence. Technologies to capture and enrich for specific target nucleotide sequences are widely available from companies like Agilent, Affymetrix, and Nimblegen and can be adopted for any target nucleotide sequence. Such a capture step may result in single stranded DNA. Hence, after the capture step, a single round of amplification, e.g. with a suitable primer, may provide for a double stranded DNA substrate.

The capture step can be performed on the target nucleotide sequence itself. In one embodiment the capture step can be performed on the same part of the target nucleotide sequence used for the introduction of the origin of amplification and selection gene. Alternatively, the capture step can be directed to another part of the target nucleotide sequence. In this respect it is observed that the target nucleotide sequence is defined herein elsewhere with a lower limit of at least 16-23 nucleotides or 10 nucleotides in the case of rare cutting restriction enzymes. The upper limit of the target nucleotide sequence can be significantly higher and can be up to 3000 nucleotides. Typically, it will not exceed 500 nucleotides, preferably 200 nucleotides, more preferably 100 nucleotides. It is in fact only limited by the available knowledge of the target sequence. In such a case, the capture can be directed to a part of the target sequence located nearby (within 100, preferably 50, more preferably 25 nucleotides) of the insertion point for the origin of amplification and selection gene

In one embodiment of the method of the invention, a guide-RNA that directs a genome-editing protein such as CRISPR-Cas9, nuclease-dead (nd-)Cas9, CRISPR-Cpf1 or nuclease-dead (nd-)Cpf1 to the target nucleotide sequence may also serve to capture and enrich for genomic fragments, ligated DNA fragments or DNA circles comprising the target nucleotide sequence. In this embodiment, the genome-editing proteins may carry an affinity tag (Histidine-tag, antibody-tag) or may carry biotin moieties that can be used to pull down the genome-editing protein associated with the genomic DNA fragments, ligated DNA fragments or DNA circles comprising the target nucleotide sequence. Alternatively, this pull down may be carried out by an antibody against said genome-editing protein. In this multistep enrichment protocol involving at least one capture step to pull down the target nucleotide sequence and one editing step to introduce the origin of replication and/or selection gene into the target nucleotide sequence, each individual step may be directed to the identical nucleotide sequence of interest. Alternatively, each step may be directed to different parts of the target nucleotide sequence. Preferably, these parts are not further than a few hundreds (100) to maximally a few thousands of basepairs (3 kb) apart on the reference sequence of the target nucleotide sequence. Furthermore, said DNA insert comprising the origin of replication sequence and/or a selection gene may also comprise a label, wherein the DNA circles comprising the DNA insert can be captured and separated using the label prior to transferring the DNA circles into the host cell. For example, suitable labels may be biotin and digoxigenin for which antibodies or affinity raisins are available that allow selection via e.g. streptavidin coated beads and beads coated with anti-digoxigenin antibodies, respectively.

In a further aspect of the invention, for multiple genomic regions of interest, each comprising a target nucleotide sequence the methods of the invention can be carried out simultaneously. Because the introduction of an origin of replication and/or a selection gene at a target nucleotide sequence can be highly selective, i.e. resulting substantially only at the target nucleotide, selection and amplification of multiple genomic regions of interest may be combined. These multiple genomic regions of interest may be genomic regions that are non-overlapping but may also be genomic regions that are overlapping each other on the reference genome. These multiple genomic regions of interest may be derived from multiple sample DNAs, e.g. from multiple subjects. These multiple genomic regions of interest may also be derived from a single sample DNA.

In another embodiment, the genomic region of interest selection and amplification method of the invention may comprise providing DNA circles with the target nucleotide sequence selectively with an origin of replication sequence and a selection gene by editing the target nucleotide sequence, wherein the selectivity is provided by using a capture probe, such as commercially provided by Agilent, Affymetrix, Nimblegen, that hybridizes to the target nucleotide sequence. Hence, in another aspect of the invention a method is provided for selection and amplification of a genomic region of interest, wherein said genomic region of interest contains a target nucleotide sequence, wherein the method comprises the steps of:
providing a sample of crosslinked DNA;
fragmenting the crosslinked DNA to provide for crosslinked DNA fragments, wherein said crosslinked DNA fragments comprise crosslinked DNA fragments containing the target nucleotide sequence;
ligating the crosslinked DNA fragments to provide for ligated crosslinked DNA fragments;
reversing the crosslinking to provide for ligated DNA fragments;
generating DNA circles derived from genomic DNA from the ligated DNA fragments.
generating DNA circles from the ligated DNA fragments, wherein DNA circles with the target nucleotide sequence are provided with an origin of replication sequence and a selection gene gene by editing the target nucleotide sequence,;
transferring the DNA circles into a host cell; and
culturing host cells comprising DNA circles with said origin of replication sequence and said selection gene;
wherein the ligated DNA fragments comprising a target nucleotide sequence are captured with a probe that hybridizes to the target nucleotide and separated from ligated DNA fragments not comprising the target nucleotide sequence, or,
wherein DNA circles comprising a target nucleotide sequence are captured with a probe that hybridizes to the target nucleotide and separated from DNA circles not comprising the target nucleotide sequence, before the step of transferring the DNA circles into a host cell or after the step of culturing the host cells.

In another embodiment, the genomic region of interest selection and amplification method of the invention may comprise providing DNA circles with the target nucleotide sequence selectively with an origin of replication sequence and a selection gene, wherein the selectivity is provided by using e.g. a capture probe, such as commercially provided by Agilent, Affymetrix, Nimblegen, that hybridizes to the target nucleotide sequence. Hence, in another aspect of the invention a method is provided for selection and amplification of a genomic region of interest, wherein said genomic region of interest contains a target nucleotide sequence, wherein the method comprises:
providing a sample of genomic DNA;
fragmenting the genomic DNA to provide genomic DNA fragments, wherein said genomic DNA fragments comprise genomic DNA fragments containing the target nucleotide sequence;
circularize the genomic DNA fragments to obtain DNA circles derived from genomic DNA, wherein said DNA circles comprise DNA circles containing the target nucleotide sequence.
generating DNA circles from the ligated DNA fragments, wherein DNA circles with the target nucleotide sequence are provided with an origin of replication sequence and a selection gene;
transferring the DNA circles into a host cell; and
culturing host cells comprising DNA circles with said origin of replication sequence and said selection gene;
wherein the ligated DNA fragments comprising a target nucleotide sequence are captured with a probe that hybridizes to the target nucleotide and separated from ligated DNA fragments not comprising the target nucleotide sequence, or,
wherein DNA circles comprising a target nucleotide sequence are captured with a probe that hybridizes to the target nucleotide and separated from DNA circles not comprising the target nucleotide sequence, before the step of transferring the DNA circles into a host cell or after the step of culturing the host cells.

Hence, in another aspect of the invention a method is provided for selection and amplification of a genomic region of interest, wherein said genomic region of interest contains a target nucleotide sequence, wherein the step of:
providing the DNA circles containing the target nucleotide sequence selectively with an origin of replication sequence and a selection gene;
comprises the steps of:
capturing with a probe that hybridizes to the target nucleotide sequence the said DNA circles, or ligated DNA fragments, or genomic DNA fragments comprising the target nucleotide sequence, and separate these from DNA circles, ligated DNA fragments or genomic DNA fragments not comprising the target sequence. This way, selectivity may be provided by the capture step and the provision of DNA circles with the origin of replication sequence and a selection gene is selective due thereto. In this embodiment it is not required to use the target nucleotide sequence for the introduction of the origin of replication and/or selection gene, as selectivity is already provided by the capture step.

Hence, in another aspect of the invention a method is provided for selection and amplification of a genomic region of interest, wherein said genomic region of interest contains a target nucleotide sequence, wherein the step of:
providing the DNA circles containing the target nucleotide sequence selectively with an origin of replication sequence and a selection gene;
comprising the steps of:
capturing with a probe that hybridizes to the target nucleotide sequence the said DNA circles, or ligated DNA fragments, or genomic DNA fragments comprising the target nucleotide sequence, and separate these from DNA circles, ligated DNA fragments or genomic DNA fragments not comprising the target sequence.

In this embodiment the capturing of DNA circles, or ligated DNA fragments or genomic DNA fragments comprising the target nucleotide sequence, serves as a first enrichment step, facilitating the subsequent introduction of the origin of replication and/or selection gene into the target nucleotide sequence.

In any case, the genomic region of interest selection and amplification methods according to the invention, rely on host cell selection and amplification. Such methods are highly useful for high throughput sequencing and microarray analysis. For example, when a method according to the invention is carried out and the selected and amplified genomic regions of interest are sequenced, a contiguous sequence of the genomic region of interest containing the target nucleotide sequence can be built. Also, sequence scaffolds can be built that combined with further sequence information allows to build contiguous sequences. Such building of genomic region of interest can occur at the level of alleles, i.e. so called haplotyping. The methods of the invention provide for highly improved haplotyping as the amplification method is much less error prone and allows for larger stretches of ligated DNA fragments to be sequenced. Hence, the methods of the invention can be utilized for building a sequence scaffold of a genomic region of interest containing a target nucleotide sequence, or for building a contiguous sequence of a genomic region of interest containing a target nucleotide sequence, or for building a haplotype of a genomic region of interest containing a target nucleotide sequence, or for determining the ploidy of a genomic region of interest containing a target nucleotide sequence

### EXAMPLES

Example of Genomic Region of Interest Amplification and Selection Using Genomic DNA Fragments

For this experiment first a gene of interest is selected (e.g.) a disease gene. In the gene of interest a target nucleotide sequence, is selected. A CRISPR guide-RNA (gRNA) molecule is designed that, when combined with the Cas9 enzyme (as available from New England Biolabs Inc., catalogue #M0386L), specifically cleaves the target nucleotide fragment. A DNA insert is prepared by PCR amplification with two primers of a DNA template comprising an origin of replication and an antibiotic resistance gene as a selection marker. Said primers comprising from 5′ to 3′, 15 (or more) nucleotides complementary to the 15 base pair sequence immediately flanking each fragment end of the Cas9 breakpoint as designed. One primer is complementary to the one fragment end, and the other primer complementary to the other fragment end as comprised in a linearized DNA circle. These are the so-called in-fusion overhangs. The primers comprise further subsequently from 5′ to 3′, an optional Notl recognition sequence and finally sequences complementary to either end of the template comprising an origin of replication and a selection marker, with one primer having a sequence complementary to one end of the template and the other primer having the sequence complementary to the other end of the said template. The DNA insert may optionally be sequenced to confirm that the template sequence is intact and flanked on either side by the Not I recognition sequence and the two in-fusion overhangs. An experiment including such a Cas9 mediated bacterial amplification strategy is for example carried out as follows:
1) a sample of fragmented genomic DNA is provided;
2) optionally, this sample is further fragmented by mechanical shearing to obtain fragments in the 30 Kb-60 Kb size range.
3) the ends of the fragments are end-repaired and blunted
4) optionally the ends are 3′A tailed and a (biotinylated) double stranded DNA linker with 3′T overhangs is added
5) ligation is carried out to circularize the fragments (with or without the insertion of the double stranded linker
6) optionally the biotinylated linkers are removed by means of size selection
7) optionally the linear DNA fragments and linear DNA linkers are removed by exonuclease treatment
8) optionally, biotinylated linker-containing DNA fragments are streptavidin purified
9) a double stranded break is induced within the DNA fragment containing the target nucleotide sequence by adding CRISPR gRNA, designed to specifically cleave the target nucleotide sequence, and the Cas9 enzyme;
10) ligation independent cloning is carried out by mixing the DNA insert and the DNA circles comprising the CRISPR/Cas9 linearized DNA circles and e.g. In-Fusion HD enzyme blend (catalogue #638909-638911, 638916-638920), as instructed by the manufacturer, In-Fusion HD Cloning Kit User Manual, Clontech 011614, Mountain View, USA.
11) optionally, the ligated DNA is purified;
12) transform the ligated DNA into (ultra-)competent bacteria;
13) culture transformed bacteria in liquid antibiotic-containing medium;
14) isolate amplified DNA circles from the cultured bacteria;
15) optionally, digest with Not I to excise the DNA insert from the DNA circles carrying the ligated Nla III fragments of interest that were ligated to the viewpoint Nla III restriction fragment, wherein the minimal backbone cassette can be removed e.g. by size selection;
16) optionally, the DNA circles (or DNA circles with DNA insert excised) are fragmented via sonication;
17) prepare a standard NGS library, including ligating sequencing adapters to the DNA templates for direct sequencing;
18) map sequence reads to a reference genome and/or build a contiguous sequence from the reads.

Example of Genomic Region of Interest Amplification and Selection Using Genomic DNA Fragments and CRISPR/Cpf1

For this experiment first a gene of interest is selected (e.g.) a disease gene. In the gene of interest a target nucleotide sequence, is selected. A CRISPR guide-RNA (gRNA) molecule is designed that, when combined with the Cpf1 enzyme, specifically cleaves the target nucleotide fragment leaving specific 5 nucleotides 5′ staggered ends (as described in Zetche et al. Cell, September 2015). A double stranded DNA insert is prepared by PCR amplification with two primers of a DNA template comprising an origin of replication and an antibiotic resistance gene as a selection marker. Said primers comprise from 5′ to 3′, an overhang comprising a T-rich protospacer-adjacent motif (PAM) that by Cpf1 cleavage enables creating 5′staggered overhangs compatible with those created at the target nucleotide sequence and sequences complementary to either end of the DNA template comprising an origin of replication and an antibiotic resistance gene as a selection marker. One primer is complementary to the one template end, and the other primer complementary to the other template end. The DNA insert may optionally be sequenced to confirm that the template sequence is intact and flanked on either side by the Not I recognition sequence and the PAM sequences. After PCR amplification, the PCR amplified template comprising an origin of replication and an antibiotic resistance gene as a selection marker is digested by Cpf1, using complementary guide RNAs that target Cpf1 to both ends of the PCR product such that 5′ staggered ends are created on both ends that are compatible with the 5′ staggered ends that Cpf1 created in the target nucleotide sequence, the An experiment including such a Cpf1 mediated bacterial amplification strategy is for example carried out as follows:
1) a sample of fragmented genomic DNA is provided;
2) optionally, this sample is further fragmented by mechanical shearing to obtain fragments in the 30 Kb-60 Kb size range.
3) the ends of the fragments are end-repaired and blunted
4) optionally the ends are 3′A tailed and a (biotinylated) double stranded DNA linker with 3′T overhangs is added
5) ligation is carried out to circularize the fragments (with or without the insertion of the double stranded linker
6) optionally the biotinylated linkers are removed by means of size selection
7) optionally the linear DNA fragments and linear DNA linkers are removed by exonuclease treatment
8) optionally, biotinylated linker-containing DNA fragments are streptavidin purified
9) a double stranded 5′ staggered break is induced within the DNA fragment containing the target nucleotide sequence by adding CRISPR gRNA, designed to specifically cleave the target nucleotide sequence, and the Cpf1 enzyme;
10) ligation is carried out by mixing the DNA insert comprising Cpf1 created compatible 5′overhangs and the DNA circles comprising the CRISPR/Cpf1 linearized DNA circles.
11) optionally, the ligated DNA is purified;
12) transform the ligated DNA into (ultra-)competent bacteria;
13) culture transformed bacteria in liquid antibiotic-containing medium;
14) isolate amplified DNA circles from the cultured bacteria;
15) optionally, digest with Not I to excise the DNA insert from the DNA circles carrying the ligated Nla III fragments of interest that were ligated to the viewpoint Nla III restriction fragment, wherein the minimal backbone cassette can be removed e.g. by size selection;
16) optionally, the DNA circles (or DNA circles with DNA insert excised) are fragmented via sonication;
17) prepare a standard NGS library, including ligating sequencing adapters to the DNA templates for direct sequencing;
18) map sequence reads to a reference genome and/or build a contiguous sequence from the reads.

A Target Locus Amplification Strategy Using a Rare Cutter and Cloning

First, a gene of interest (e.g. a disease gene) is selected. In or near the gene of interest, a restriction enzyme recognition site is selected in or near the gene of interest (preferably within 20 kilobases) that is a rare cutter, i.e. an enzyme that recognizes at least 6 nucleotides. Preferably said selected restriction enzyme recognition site has 8 or more base pairs (e.g. Not I). When e.g. an Nla III restriction enzyme is used for fragmenting the crosslinked DNA, the Nla III restriction fragment that contains the Not I restriction site of interest corresponds to the target nucleotide sequence. The Not I restriction site and the at least 15 nucleotides flanking each side of the Not I restriction site can also be regarded to correspond to the target nucleotide sequence, e.g. when a different fragmenting step is used. A DNA insert is prepared by PCR amplification with two primers of a DNA template comprising an origin of replication and an antibiotic resistance gene as a selection marker.

The primers comprise from 5′>3′, 15 (or more) nucleotides complementary to 15 nucleotides flanking each side of the Not I restriction site of interest, with one primer complementary to the one fragment end, and the other primer complementary to the other fragment end (these are the LiC overhangs), the restriction enzyme recognition sequence (i.e. in this example Not I), sequences complementary to either end DNA template, with the one primer having the sequence complementary to the one DNA template end and the other primer having the sequence complementary to the other DNA template end. The inclusion of enzyme restriction sites, which is optional and is in this case Not I, allows for removal of the origin of replication and/or selection marker gene with a digest and self-ligation. The DNA insert can be analysed by Sanger sequencing to confirm that the DNA insert has the features as designed. A targeted amplification strategy can be carried out as follows.
1) a sample of crosslinked DNA is provided;
2) the crosslinked DNA is fragmented, e.g. with Nla III;
3) the fragmented crosslinked DNA is ligated to create DNA circles;
4) the crosslinking is reversed to obtained ;
5) the DNA circles are digested with e.g. Not I to provide for linearized DNA molecules;
6) in fusion ligation is carried out by mixing the DNA insert and the Not I linearized DNA molecule and adding e.g. e.g. In-Fusion HD enzyme blend (catalogue #638909-638911, 638916-638920), as instructed by the manufacturer, In-Fusion HD Cloning Kit User Manual, Clontech 011614, Mountain View, USA.
7) optionally purify the DNA;
8) the DNA is transformed into (ultra-)competent bacteria;
9) the transformed bacteria are cultured in antibiotic-containing medium;
10) DNA is isolated from the bacterial culture;
11) optionally, the DNA is digested with Not I to excise the DNA insert from DNA circles, e.g. via size selection;
12) optionally, the inserts are fragmented via sonication
13) a standard NGS library is prepared, sequencing adapters are ligated to the template for direct sequencing;
14) reads are e.g. mapped to a reference genome and/or a contig is built from the reads

Targeted Amplification Strategy Using Achilles Cleavage and Coning

In this approach first a gene of interest (e.g. a disease gene) is selected. Next, a restriction site is selected that can be methylated by methyltransferases such as Hhall (Hinfl) methyltransferase (MH-Hhall), DAM or DCM, in or near (preferably within 20 kb) the gene of interest. A restriction site and sequences flanking this restriction site is selected as the targeting sequence, e.g. a Nla III fragment containing this restriction site. Using an Achilles Cleavage reaction, or the like, such as described in Koob et al., 1992 Nov. 11; 20(21):5831-6. In such a method, a probe (or probes) are designed to hybridize with the target nucleotide sequence such that the target nucleotide sequence can be selectively protected from DNA methylation. Upon subsequent digestion of the restriction site of interest with a corresponding methylation-sensitive restriction enzyme, only the target nucleotide sequence will be restricted. A DNA insert is prepared by PCR amplification with two primers of a DNA template comprising an origin of replication and an antibiotic resistance gene as a selection marker. The primers comprise from 5′>3′, 15 (or more) nucleotides complementary to 15 nucleotides flanking each side of the Not I restriction site of interest, with one primer complementary to the one fragment end, and the other primer complementary to the other fragment end (these are the in-fusion overhangs), a restriction enzyme recognition sequence (e.g. a rare cutter, in this example Not I), sequences complementary to either end DNA template, with the one primer having the sequence complementary to the one DNA template end and the other primer having the sequence complementary to the other DNA template end. The DNA insert can be analysed by Sanger sequencing to confirm that the DNA insert has the features as designed. A targeted amplification strategy can be carried out as follows.
1) a sample of crosslinked DNA is provided;
2) the crosslinked DNA is fragmented with e.g. a restriction enzyme such as Nla III;
3) the fragmented crosslinked DNA is ligated to create DNA circles;
4) the crosslinking is reversed;
5) an Achilles' Cleavage (AC) reaction is carried out to site-specifically open the DNA circles containing the viewpoint Nla III restriction fragment at the restriction site of interest;
6) an in-fusion ligation reaction is carried out by mixing the DNA insert and the Achilles' cleaved linear DNA and performing e.g. e.g. In-Fusion HD enzyme blend (catalogue #638909-638911, 638916-638920), as instructed by the manufacturer, In-Fusion HD Cloning Kit User Manual, Clontech 011614, Mountain View, USA.
7) optionally, purify the DNA
8) transform the DNA into (ultra-)competent bacteria
9) transformed bacteria are cultured in antibiotic-containing medium;
10) isolate DNA from the bacteria;
11) optionally, digest the DNA with Not I to excise the DNA insert, and e.g. remove the DNA insert by size selection;
12) optionally, the inserts are fragmented via sonication
13) carry out standard NGS library preparation: sequencing adapters are ligated to the template for direct sequencing;
14) map reads e.g. to the reference genome and/or build a contig from reads

Example of a PCR Bacterial Amplification Strategy Combined with Cloning

An enrichment step of DNA fragments ligated to the DNA fragment comprising the target nucleotide sequence via limited PCR prior to the cloning step may be contemplated. First, a gene of interest (e.g. a disease gene) is selected and a target nucleotide sequence is selected in or near (preferably within 20 kb) the gene of interest, e.g. a restriction fragment such as an Nla III fragment. Inverse primers are designed that bind to the target nucleotide sequence. A DNA insert is prepared by PCR amplification with two primers of a DNA template comprising an origin of replication and an antibiotic resistance gene as a selection marker, said primers comprising from 5′>3′, 15 (or more) nucleotides complementary to the 15 nucleotide sequence identical to the 5′-end bases of reverse complement of the inverse viewpoint primers, these are the in-fusion overhangs compatible with the inverse PCR product, a unique (e.g. Not I) recognition sequence, and sequences complementary to either end of the minimal backbone cassette, with the one primer having the sequence complementary to the one end of the DNA insert and the other primer having the sequence complementary to the other end of the DNA insert. The DNA insert can be analysed by Sanger sequencing, to confirm that the sequence is as designed. An experiment combining PCR amplification combined with cloning can be carried out as follow:
1) a sample of crosslinked DNA is provided;
2) the crosslinked DNA is fragmented with e.g. Nla III;
3) the fragmented crosslinked DNA is ligated to create DNA circles;
4) the crosslinking is reversed;
5) a restriction is carried out with a secondary restriction enzyme, for example Nspl;
6) the secondary digested sample is ligated to obtain DNA circles;
7) the DNA circles obtained in step 6) are PCR amplified using the inverse primers specific for the target nucleotide sequence, preferably with e.g. less than 10 amplification cycles;
8) ligation independent cloning is performed by mixing the DNA insert and the amplified template obtained in step 7) and adding e.g. In-Fusion HD enzyme blend (catalogue #638909-638911, 638916-638920), as instructed by the manufacturer, In-Fusion HD Cloning Kit User Manual, Clontech 011614, Mountain View, USA.
9) optionally, purify the DNA
10) transform the DNA into (ultra-)competent bacteria;
11) culture the transformed bacteria in antibiotic-containing medium;
12) isolate DNA from the bacteria
13) optionally, digest the isolated DNA with Not Ito excise the DNA insert, and e.g. remove the DNA insert by size selection;
15) optionally, the inserts are fragmented via sonication
16) prepare a standard NGS library, sequencing adapters are ligated to the template for direct sequencing;
17) map the reads e.g. to the reference genome and/or build a contiguous sequence from the reads.

Cas-9 Mediated Bacterial Amplification Strategy:

For this experiment first a gene of interest is selected (e.g.) a disease gene. In the gene of interest a target nucleotide sequence, e.g. an Nla III fragment, within or close to the gene of interest (for example within 20 Kb) is selected. A CRISPR guide-RNA (gRNA) molecule is designed that, when combined with the Cas9 enzyme, specifically cleaves the target nucleotide fragment, leaving e.g. at least 15 basepairs between the breakpoint and the e.g. Nla III sites flanking the target nucleotide sequence as described e.g. in Li, et al. Nat Methods, 2007. 4(3): p. 251-6. and Zhang, et al. Methods Mol Biol, 2014. 1116: p. 235-44. A DNA insert is prepared by PCR amplification with two primers of a DNA template comprising an origin of replication and an antibiotic resistance gene as a selection marker. Said primers comprising from 5′ to 3′, 15 (or more) nucleotides complementary to the 15 base pair sequence immediately flanking each fragment end of the Cas9 breakpoint as designed. One primer is complementary to the one fragment end, and the other primer complementary to the other fragment end of the Nla III fragment as comprised in a linearized DNA circle. These are the so-called in-fusion overhangs. The primers comprise further subsequently from 5′ to 3′, an optional Not I recognition sequence and finally sequences complementary to either end of the template comprising an origin of replication and a selection marker, with one primer having a sequence complementary to one end of the template and the other primer having the sequence complementary to the other end of the said template. The DNA insert may optionally be sequenced to confirm that the template sequence is intact and flanked on either side by the Not I recognition sequence and the two in-fusion overhangs. An experiment including such a Cas9 mediated bacterial amplification strategy is for example carried out as follows:
1) a sample of crosslinked DNA is provided;
2) the crosslinked DNA if fragmented with eg Nla III;
3) the fragmented crosslinked DNA to is ligated create DNA circles;
4) the crosslinking is reversed to obtain DNA circles;
5) a double stranded break is induced within the DNA fragment containing the target nucleotide sequence by adding both CRISPR gRNAs, designed to specifically cleave the at least 15 base pair sequence, and the Cas9 enzyme;
6) ligation independent cloning is carried out by mixing the DNA insert and the DNA circles comprising the CRISPR/Cas9 linearized DNA circles and adding e.g. In-Fusion HD enzyme blend (catalogue #638909-638911, 638916-638920), as instructed by the manufacturer, In-Fusion HD Cloning Kit User Manual, Clontech 011614, Mountain View, USA.
7) optionally, the ligated DNA is purified;
8) transform the ligated DNA into (ultra-)competent bacteria;
9) culture transformed bacteria in liquid antibiotic-containing medium;
10) isolate amplified DNA circles from the cultured bacteria;
11) optionally, digest with Not I to excise the DNA insert from the DNA circles carrying the ligated Nla III fragments of interest that were ligated to the viewpoint Nla III restriction fragment, wherein the minimal backbone cassette can be removed e.g. by size selection;
12) optionally, the DNA circles (or DNA circles with DNA insert excised) are fragmented via sonication;
13) prepare a standard NGS library, including ligating sequencing adapters to the DNA templates for direct sequencing;
14) map sequence reads to a reference genome and/or build a contiguous sequence from the reads.

1. A method for selection and amplification of a genomic region of interest, wherein the genomic region of interest contains a target nucleotide sequence, wherein the method comprises the steps of:
providing DNA circles derived from genomic DNA, wherein one or more DNA circles contain the target nucleotide sequence;
selectively inserting an origin of replication sequence and a selection gene into the DNA circles containing the target nucleotide sequence by;
i) linearizing the DNA circles by introducing a double-stranded break at the target nucleotide sequence, resulting in a linear DNA molecule with fragment ends derived from the target nucleotide sequence;
ii) providing a DNA insert comprising the origin of replication sequence and the selection gene;
iii) allowing the linear DNA molecule and the DNA insert to form a DNA circle containing the origin of replication sequence and the selection gene;
transferring the DNA circles containing the origin of replication sequence and the selection gene into host cells; and
selectively culturing the host cells comprising DNA circles containing the origin of replication sequence and the selection gene.

2. The method according to claim 1, wherein the double stranded break is selectively introduced at the target nucleotide sequence by using at least one of
a restriction enzyme with a restriction recognition site of at least 8 nucleotides;
an Achilles Cleavage method; and
a sequence-specific genome editing nuclease.

3. The method according to claim 1, wherein the sequence-specific genome editing nuclease is at least one of CRISPR-Cas9 and CRISPR-Cpf1.

4. The method according to claim 1, wherein the step of providing DNA circles derived from genomic DNA comprises the steps of:
providing a sample of genomic DNA;
fragmenting the genomic DNA to provide genomic DNA fragments, wherein one or more genomic DNA fragments comprise the target nucleotide sequence; and
circularizing the genomic DNA fragments to obtain DNA circles, wherein one or more DNA circles comprise the target nucleotide sequence.

5. The method according to claim 4, wherein the genomic DNA is fragmented by at least one of sonication, shearing and enzymatic restriction.

6. The method according to claim 4, wherein the size of the genomic DNA fragments is in the range of 10 kb-300 kb.

7. The method according to claim 1, wherein the step of selectively inserting the origin of replication sequence and/or the selection gene comprises a method selected from the group consisting of:
homologous recombination;
restriction and ligation; and
ligation independent cloning.

8. The method according to claim 1, wherein the step of selectively inserting the origin of replication sequence and/or the selection gene comprises Gibson Assembly Cloning.

9. The method according to claim 1, wherein the genomic DNA is obtainable from a human subject.

10. The method according to claim 9, wherein the genomic DNA is obtainable from a human subject who may be at risk or suspected of having a disease or trait.

11. The method according to claim 1, wherein for multiple genomic regions of interest, each comprising a target nucleotide sequence, the method is carried out simultaneously.

12. The method according to claim 1, wherein the said host cell is a yeast cell or a bacterial cell.

13. The method according to claim 12, wherein the bacterial cell is an (ultra)competent bacterial cell.

14. The method according to claim 1, wherein the selection gene is selected from the group consisting of an antibiotic resistance gene, a pigment gene, a fluorescent gene and a host cell surface protein gene.

15. The method according to claim 1, wherein the method is for use in:
building a sequence scaffold of a genomic region of interest containing a target nucleotide sequence;
building a contiguous sequence of a genomic region of interest containing a target nucleotide sequence;
building a haplotype of a genomic region of interest containing a target nucleotide sequence; or
determining the ploidy of a genomic region of interest containing a target nucleotide sequence.

16. The method according to claim 6, wherein the size of the genomic DNA is in the range of 20 kb-100 kb.

17. The method according to claim 6, wherein the size of the genomic DNA is in the range of 30-60 kb or wherein the size of the genomic DNA fragments is in the range of 1-20 kb.

18. The method according to claim 10, wherein the target nucleotide sequence is associated with said disease or trait.
